(19) United States
(12) Patent Application Publication
(10) Pub. No.: US 2010/0144860 A1
(43) Pub. Date: Jun. 10, 2010

(54) Transglutaminase Inhibitor Comprising EGCG And A Method For Producing Thereof

(76) Inventors: Soo Youl Kim, Seoul (KR); Kyung Chae Jeong, Gyeonggi-do (KR)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/663,360

(22) PCT Filed: Jun. 11, 2008
(86) PCT No.: PCT/KR08/03265

§ 371 Date: Dec. 29, 2009

(30) Foreign Application Priority Data
Jun. 11, 2007 (KR) 10-2007-0056812

(51) Int. Cl. A61K 31/352; C07D 311/62; A61P 35/00; A61P 25/00; A61P 25/28; A61P 25/16

(52) U.S. Cl. 514/456; 549/399

(57) ABSTRACT

The present invention relates to a transglutaminase inhibitor comprising epigallocatechin gallate (hereinafter, referred to as EGCG). More particularly, the present invention relates to a transglutaminase inhibitor comprising EGCG which effectively inhibits the activity of transglutaminase, the overexpression of which is responsible for the etiology of various diseases, and to novel uses thereof. According to the present invention, provided is a transglutaminase inhibitor and a method of inhibiting transglutaminase, featuring the use of EGCG as an active ingredient. Featuring the use of EGCG, the novel method of inhibiting transglutaminase according to the present invention is safely applied to patients who suffer from the diseases caused by the overexpression of transglutaminase, thereby obtaining an inhibitory effect against transglutaminase without casuing side-effects.

### TECHNICAL FIELD

The present invention relates to a transglutaminase inhibitor comprising epigallocatechin gallate (EGCG). More particularly, the present invention relates to a transglutaminase inhibitor comprising EGCG which effectively inhibits the activity of transglutaminase, the overexpression of which is responsible for the etiology of various diseases, and to novel uses thereof.

### BACKGROUND ART

Transglutaminases are protective enzymes which are responsible for blood clotting in response to tissue injury under normal conditions. However, these enzymes are also reported to play an important role in the pathological mechanism of various diseases in the absence of regulatory-control in the level of expression thereof (review article. Soo-Youl Kim: New Target Against Inflammatory Diseases: Transglutaminase 2. Archivum Immunologiae & Therapiae Experimentalis 52, 332-337, 2004).

The expression of transglutaminases increases particularly upon the occurrence of various inflammatory diseases, including rheumatoid arthritis, diabetes, inflammatory myositis, atherosclerosis, stroke, liver cirrhosis, breast cancer, Alzheimer's disease, Parkinson's disease, Huntington's disease, encephalitis, and celiac disease. Also, transglutaminases are observed to increase in expression level, along with NF-κB, when cancer enters metastasis or changes into chemo-resistance or radio-resistance (Soo-Youl Kim. Transglutaminase 2 in inflammation. Front Biosci. 11, 3026-3035, 2006).

The relationship between transglutaminases and chemo-resistance in cancer has remained unclear so far. However, when the expression of transglutaminases was suppressed in chemoresistant breast cancer cells, the cancer cells were getting highly susceptible to chemicals, and finally died (Antonyak et al., Augmentation of tissue transglutaminase expression and activation by epidermal growth factor inhibit doxorubicin-induced apoptosis in human breast cancer cells. J Biol Chem. 2004 Oct. 1;279(40):41461-7.; Dae-Seok Kim et al. Reversal of Drug Resistance in Breast Cancer Cells by Transglutaminase 2 Inhibition and Nuclear Factor-KB Inactivation. Cancer Res. 2006. in press).

Also, there is a strong reason for suppressing the activity of transglutaminases as the etiological mechanism for which the activation of transglutaminases is responsible is elucidated at the molecular level (Key Chung Park, Kyung Cheon Chung, Yoon-Seong Kim, Jongmin Lee, Tong H. Joh, and Soo-Youl Kim. Transglutaminase 2 induces nitric oxide synthesis in BV-2 microglia. Biochem. Biophys. Res. Commun. 323, 1055-1062, 2004; Jongmin Lee, Yoon-Seong Kim, Dong-Hee Choi, Moon S. Bang, Tay R. Han, Tong H. Joh, and Soo-Youl Kim. Transglutaminase 2 induces NF-KB activation via a novel pathway in BV-2 microglia. J. Biol. Chem. 279, 53725-53735, 2004; Dae-Seok Kim et al. Reversal of Drug Resistance in Breast Cancer Cells by Transglutaminase 2 Inhibition and Nuclear Factor-KB Inactivation. Cancer Res. 2006. in press).

Inflammation is largely attributable to NF-κB activation. NF-κB is known to be activated by kinases in signal transduction pathways. However, NF-κB was also found to be activated independently of kinases, thereby negating the function of kinase inhibitors (Tergonkar et al., IkappaB kinase-independent IkappaBalpha degradation pathway: functional NF-kappaB activity and implications for cancer therapy. Mol Cell Biol. 2003 Nov;23(22):8070-83.).

In a previous study conducted by the present inventors, it was reported that transglutaminase activates NF-κB independently of the activation of linases (IKK, NAK), by inducing crosslinking I-κBa (Jongmin Lee, et al. Transglutaminase 2 induces NF-kB activation via a novel pathway in BV-2 microglia. J. Biol. Chem. 279, 53725-53735, 2004). Transglutaminases are calcium-dependent enzymes, which can activate NF-κB only at an elevated intracellular level of calcium.

Upon inflammation, the activation of the transcriptional factor NF-κB leads to an increase in the expression not only of inflammatory factors including transglutaminases, but also of its inhibitor 1-κBα. Continuous NF-κB activation is inhibited by 1-κBα under normal conditions, but continues in chronic inflammatory diseases. Interestingly, TNF-α or LPS (lipopolysaccharide)-induced NF-κB activation gives rise to transglutaminase expression. Thus, aberrantly activated transglutaminases in inflammatory cells are expected to activate NF-κB directly or to further maintain activated NF-κB, thereby playing a key role in inflammation maintenance (FIG. 1). In addition, this vicious cycle may be a main cause of cancer metastasis and chemoresistance (Jongmin Lee, et al. Transglutaminase 2 induces NF-κB activation via a novel pathway in BV-2 microglia. J. Biol. Chem. 279, 53725-53735, 2004).

Therefore, a transglutaminase inhibitor may play a crucial role in breaking the continuous cycle of NF-κB, on which the steroid-substituting effect proposed by the present inventors is based (Sohn, J., Kim, T.-I., Yoon, Y.-H., and Kim, S.-Y.: Transglutaminase Inhibitor: A New Anti-Inflammatory Approach in Allergic Conjunctivitis. J. Clin. Invest. 111, 121-8, 2003).

Amine compounds are known to inhibit transglutaminase activity. Representative of the transglutaminase inhibitors are cystamine (nature Genetics 18, 111-117, 1998; Nature Medicine 8, 143-149, 2002) and putrescine. In addition to the amine compounds, other chemical inhibitors, such as monodansylcadaverine (J. Med. Chem. 15, 674-675, 1972), w-dibenzylaminoalkylamine (J. Med. Chem. 18, 278-284, 1975), 3-halo-4,5-dihydroisoxazole (Mol. Pharmacol. 35, 701-706, 1989), and 2-[(2-oxopropyl)thio]imidazolium derivatives (Blood, 75, 1455-1459, 1990), were developed, but are reported to be so toxic as to non-specifically inhibit other enzymes in vivo.

Therefore, there is a need for safe and effective transglutaminase-specific inhibitors. Recently, Sohn et al. have succeeded in obtaining the same effect from recombinant peptides as steroidal drugs for the inflammation of allergic conjunctivitis to ragweed in a guinea pig model (Sohn, J., Kim, T.-I., Yoon, Y.-H., and Kim, S.-Y.: Transglutaminase Inhibitor: A New Anti-Inflammatory Approach in Allergic Conjunctivitis. J. Clin. Invest. 111, 121-8, 2003). In this regard, anti-flammin protein (PLA2 inhibitor) or elafin protein (very strong transglutaminase substrate, Nara, K., et al. 1994. Elastase inhibitor elafin is a new type of proteinase inhibitor which has a transglutaminase-mediated anchoring sequence termed “cementoin”. J Biochem (Tokyo). 115:441-448)-derived synthetic peptides which mimic the catalytic site of transglutaminase were used. The expression of transglutaminases increases particularly upon the occurrence of various inflammatory diseases, including degenerative arthritis, diabetes, autoimmune myositis, arteriosclerosis, cerebral apoplexy, hepatocirrhosis, malignant breast cancer, meningitis, and inflammatory gastric ulcer.

In addition to the above-mentioned compounds, other chemical inhibitors were developed, but are reported to be so toxic as to non-specifically inhibit other enzymes. Effective as they are in inhibiting transglutaminase, peptide inhibitors developed prior to the present invention (Korean Patent Application No. 10-2006-98921) still have a lot of problems awaiting solutions in terms of production cost and safe practice.

On the other hand, (-)-epigallocatechin gallate (EGCG) is a type of polyphenol among active ingredients which are contained in Camellia sinensis belonging to the family of Theaceae, and is the most active major ingredient, and primarily responsible for the green tea effect. EGCG possesses two triphenolic groups in its structure, which are thought to be important for its stronger pharmacological action (Matsuo, N. et al., Allergy, 52(1997) 58-64). It is known that EGCG possesses strong antioxidant (Guo. G. et al., Biochim. Biophys, Acta, 1304(1996) 210-222), anti-microbial, and anti-mutagenic activities.

In addition, EGCG can be used for inhibiting MMP-9 expression and osteoclast formation, and used for preventing or treating neuronal damage induced by global ischemia, as disclosed in Korean Patent Publication Nos. 10-2005-45770 and 10-2002-55735, respectively. Disclosed are the uses of EGCG as an acetyl-cholinesterase inhibitor (Korean Patent No. 10-540369), and as an active ingredient for treating rheumatoid arthritis (Korean Patent No. 10-601080). Also, disclosed is a cosmetic composition using EGCG derivatives which are prepared by reacting EGCG with nicotinic acids (Korean Patent No. 10-449228). As mentioned above, EGCG can be applied to various fields for various purposes. However, there is no mention in the prior art that EGCG has an inhibitory activity on transglutaminase, thereby being used for treating diseases caused by the activation of transglutaminase as its inhibitor.

### DISCLOSURE OF INVENTION

### Technical Problem

Accordingly, the present inventors have conducted intensive and thorough research into transglutaminase inhibitors by screening naturally occurring compounds which have been recognized as being safe for commercialization. They found that EGCG has a potent inhibitory activity against transglutaminase, thereby completing the present invention.

### Technical Solution

It is an object of the present invention to provide a transglutaminase inhibitor comprising EGCG.

In addition, it is another object of the present invention to provide a method of inhibiting transglutaminase, featuring the use of EGCG as an active ingredient.

It is still another object of the present invention to provide a pharmaceutical composition for the treatment of diseases caused by the activation of transglutaminase, comprising EGCG.

It is still another object of the present invention to provide a method of treating diseases caused by the activation of transglutaminase, featuring the use of EGCG.

### Advantageous Effects

According to the present invention, provided is a transglutaminase inhibitor and a method of inhibiting transglutaminase, featuring the use of EGCG as an active ingredient.

Featuring the use of EGCG, the novel method of inhibiting transglutaminase according to the present invention is safely applied to patients who suffer from the diseases caused by the overexpression of transglutaminase, thereby obtaining an inhibitory effect against transglutaminase without causing side-effects.

### BRIEF DESCRIPTION OF THE DRAWINGS

FIG. 1 is a graph showing the result of in vitro assay for inhibitory effect of EGCG on transglutaminase, in which the transglutaminase-catalyzed reaction between [1,4,-¹⁴C] putrescine and succinylated casein is measured, resulting in that EGCG, competing with putrescine, acts to inhibit the activity of transglutaminase.

### BEST MODE FOR CARRYING OUT THE INVENTION

In accordance with an aspect, the present invention relates to a transglutaminase inhibitor comprising EGCG, and a method of inhibiting transglutaminase, featuring the use of EGCG as an active ingredient.

As mentioned above, epigallocatechin gallate (EGCG) is generally present in Camellia sinensis or the like, and has a molecular formula of C₂₂H₁₈O₁₁, a chemical name of [2R, 3R]-2[3,4,5-trihydroxyphenyl]-3,4-dihydro-1[2H]-benzopyran-3,5,7-triol-3-[3,4,5-trihydroxybenzonate], and a following structural formula 1.

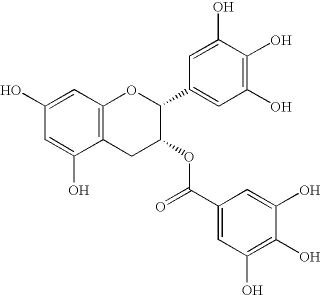

In the present invention, EGCG derived from a natural source, such as green tea, may be used, and obtained by extraction and purification according to a known method. There are various methods of preparing EGCG, such as a method using a porous polar packing material, a method using HPLC, an isolation method by adding caffeine, a method using a polyamide packing material, and a method using electron transfer (US Patent Publication No. U.S. Pat. No. 4,613,672, Korean Patent Publication No. 10-2001-50080, N. S. Kumar et al., J. Chromatogr. A 1083(2005) 223-228, D. Labbe et al., Journal of Membrane Science 254(2005) 101-109, and J. I. Kim et al., J. Chromatogr. A 949(2002) 275-280). In the present invention, EGCG may be prepared by such a known method, selected by those skilled in the art. In addition, EGCG may be directly prepared or commercially available.

In addition, EGCG is unstable and hydrophilic. Thus, if necessary, it may be stabilized, while maintaining its efficacy. The stabilization may be performed by a physical method such as inclusion using a polymer, and a chemical method such as modification into more stable derivatives, which are well known in the art. In particular, achieving improvement in both stability and solubility, the chemical method has been extensively studied. For example, 8 hydroxyl groups of EGCG may be entirely or partially substituted with an alkyl or acyl group. Therefore, EGCG used in the present invention includes EGCG extracted and purified from a natural source, as well as EGCG derivatives which are more stabilized by the above method.

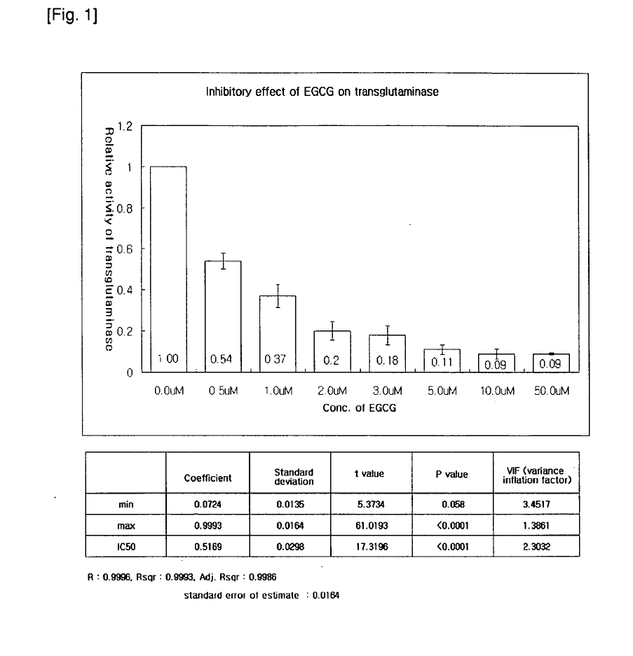

In a specific embodiment of the present invention, the present inventors measured transglutaminase-catalyzed reaction between [1,4,-¹⁴C] putrescine and succinylated casein. It was found that EGCG, competing with putrescine, acts to inhibit the activity of transglutaminase. In in vitro experiments for transglutaminase-catalyzed reaction between ¹⁴C-labelled putrescine and succinylated casein, it was found that a higher concentration of EGCG leads to a poorer activity of transglutaminase (FIG. 1). Consequently, it can be seen that EGCG is a transglutaminase inhibitor, and EGCG can reduce the increased activity of transglutaminase, even upon the overexpression of transglutaminase.

In accordance with another aspect, the present invention relates to a pharmaceutical composition for the prevention and treatment of diseases caused by the activation of transglutaminase, comprising EGCG, and to a method of treating the diseases using EGCG.

The term “prevention” as used herein means all of the actions in which the occurrence of any disease caused by the activation of transglutaminase is restrained or retarded by the administration of the pharmaceutical composition containing EGCG. The term “treatment” as used herein means all of the actions in which any disease caused by the activation of transglutaminase has taken a turn for the better or been modified favorably by the administration of the pharmaceutical composition.

In the present invention, the diseases caused by the activation of transglutaminase include all diseases that are incurred as transglutaminase activity increases, for example, upon the overexpression of transglutaminase, and are particularly exemplified by neurological diseases and cancers.

The transglutaminase peptide inhibitors are effective in the prevention or treatment of all diseases that are caused as transglutaminases are inappropriately activated. More specifically, the transglutaminase peptide inhibitors can be used for the prevention or treatment of neurolcgical diseases or cancers which are caused by inappropriate transglutaminase activation.

In accordance with still another aspect, the present invention relates to a transglutaminase-inhibiting composition, comprising at least one of the peptides of the present invention.

Typical of neurological diseases are central nervous system diseases, which are associated with the death or injury of the central nervous system, such as Alzheimer's disease, multi-infarct dementia, a mixed Alzheimer/multi-infarct dementia, Parkinson's disease, hypothyroidism, alcohol-related dementia, and Huntington's diseases. These diseases are characterized by confusion, disorientation and personality disintegration with main syndromes of cognitive dysfunction, language impairment, dysfunctions in judgment, inference, temporal and spatial adaptation and learning, finally leading to the death of afflicted patients. Of them, the diseases caused by the activation of transglutaminase, e.g., the overexpression of transglutaminase in nerve tissues, are targets of the pharmaceutical composition according to the present invention. Particularly, the pharmaceutical composition of the present invention is useful in the treatment of Huntington's disease, which is associated with the over-expression of transglutaminase in the brain (Nature Medicine, Vol 8. Number 2 , February 2002 pp 143-149), Alzheimer's disease, which is associated with the over-expression of transglutaminase in the cerebellum and cerebral cortex (The Journal of Biological Chemistry, Vol. 274. No. 43. Issue Of October 22, pp 30715-30721), and Parkinson's disease, which is associated with transglutaminase-induced α synuclein aggregation (PNAS, Feb. 18, 2003, Vol. 100, no.4, pp 2047-2052), but are not limited thereto. The present invention is applicable to the treatment of all diseases caused by the overexpression of transglutaminase in nerve tissues.

As for cancers, these are found to significantly increase in the level of expression of transglutaminase upon metastasis or entry into chemo- or radio-resistance. Thus, the suppression of transglutaminase arises as a key in the prevention and treatment of cancers. Concrete examples of the cancers, which can be prevented or treated using the pharmaceutical composition containing EGCG of the present invention, include large intestine cancer, small intestine cancer, rectal cancer, anal cancer, esophageal cancer, pancreatic cancer, stomach caner, kidney cancer, uterine cancer, breast cancer, lung cancer, lymphoma, thyroid cancer, prostatic carcinoma, leukemia, skin cancer, colon cancer, encephaloma, bladder cancer, ovarian cancer, and gallbladder carcinoma, but are not limited thereto.

The composition comprising EGCG and the treatment method of the present invention can be applied to mammals that may suffer from diseases due to the activation of transglutaminase, including cattle, horses, sheep, pigs, goats, camels, antelopes, dogs, and cats, as well as humans.

The pharmaceutical composition comprising EGCG of the present invention may be used alone or in combination with other pharmaceutical compositions.

The pharmaceutical composition comprising EGCG may be formulated into various dosage forms. For example, it may be loaded into a capsule containing EGCG without an excipient or together with a fine solid carrier and/or a liquid carrier. If necessary, the resultant may be molded into preferred formulations. Examples of the suitable carriers include starch, water, brine, ethanol, glycerol, Ringer's solution, and dextrose solutions. Reference may be made to the literature (Remington's Pharmaceutical Science, 19^{th} Ed., 1995, Mack Publishing Company, Easton Pa.) upon formulation of the pharmaceutical composition.

The pharmaceutical composition comprising EGCG of the present invention may be prepared into any dosage form, either oral or non-oral formulation, containing chlorogenic acid as an active ingredient. Non-oral dosage forms may be injections, coatings, and sprays such as aerosols, with preference for injections or sprays such as aerosols. Also preferable are oral dosage forms.

Examples of the oral dosage forms suitable for the pharmaceutical composition of the present invention include tablets, troches, lozenges, aqueous or emulsive suspensions, powder, granules, emulsions, hard or soft capsules, syrups, and elixirs. For formulation such as tablets and capsules, useful are a binder such as lactose, saccharose, sorbitol, mannitol, starch, amylopectin, cellulose and gelatin, an excipient such as dicalcium phosphate, a disintegrant such as corn starch and sweet potato starch, a lubricant such as magnesium stearate, calcium stearate, sodium stearylfumarate, and polyethylene glycol wax, a sweetener such as sucrose and saccharine, and a flavoring agent such as peppermint, methyl salicylate, and fruit flavors. For capsules, a liquid carrier such as polyethylene glycol and lipid may be further used in addition to the above-mentioned compounds.

For non-oral administration, the pharmaceutical composition of the present invention may be formulated into injections for subcutaneous, intravenous, or intramuscular routes, suppositories, or sprays inhalable via the respiratory tract, such as aerosols. Injection preparations may be obtained by dissolving or suspending EGCG, together with a stabilizer or a buffer, in water and packaging the solution or suspension in ampules or vial units. Suppositories are typically made of a suppository base, such as cocoa butter and another glyceride, or a therapeutic laxative. For sprays, such as aerosol, a propellant for spraying a water-dispersed concentrate or wetting powder may be used in combination with an additive.

The above mentioned pharmaceutically acceptable additive or carrier may include any additive or carrier which is pharmaceutically pure, substantially non-toxic, and does not interfere with the action of the active ingredient.

The pharmaceutical composition of the present invention may be administered via typical routes, such as rectal, local, intravenous, intraperitoneal, intramuscular, intraarterial, transdermal, intranasal, inhalational, intraocular, and subcutaneous routes. Non-oral administration means administration modes including intravenous, intramuscular, intraperitoneal, intrasternal, transdermal and intraarterial routes. For administration via non-oral routes, the pharmaceutical composition comprising EGCG in a desired purity is preferably mixed with a pharmaceutically acceptable carrier, that is, a carrier being non-toxic at dosage concentrations and amounts, and compatible with other ingredients, and then formulated into a unit dosage form. In particular, it is required to exclude oxidants and other compounds known to be hazardous to the human body.

The EGCG of the present invention may be administered along with at least one pharmaceutically acceptable excipient as a pharmaceutical composition. It will be obvious to those skilled in the art that when the pharmaceutical composition of the present invention is administered to human patients, the total daily dose should be determined through appropriate medical judgment by a physician. The therapeutically effective amount for patients may vary depending on various factors well known in the medical art, including the find and degree of the response to be achieved, concrete compositions according to whether other agents are used therewith or not, the patient's condition such as age, body weight, state of health, sex, and diet, the frequency, time and route of administration, the secretion rate of the composition, the time period of therapy, etc. For agents suitable for use in the art, reference may be made to the literature (Remington's Pharmaceutical Science, 19^{th}Ed., 1995, Mack Publishing Company, Easton Pa.). Accordingly, the effective dosage of EGCG is preferably determined with reference to the above-mentioned considerations. The pharmaceutical composition of the present invention may be administered in an effective amount of EGCG of 1 to 2000 mg.

Hereinafter, a better understanding of the present invention may be obtained through the following examples which are set forth to illustrate, but are not to be construed as the limit of the present invention.

### MODE FOR THE INVENTION

### Example 1

### In vitro Assay for Inhibition of Transglutaminase Activity

To measure the inhibitory activity of EGCG which competes with putrescine, the transglutaminase-catalyzed reaction between [1,4,-¹⁴C] putrescine and succinylated casein were observed.

Succinylated casein was purchased from Calbiochem (Cat. No. 573464), and 1 g of the powder was dissolved in 50 ml of a reaction buffer solution containing 5 mM DTT (0.1 M Tris-acetate (pH 8.0), 10 mM CaCl₂, 0.15 M NaCl, 1.0 mM EDTA). This solution was stored in a deep freezer for further use. [1,4-¹⁴C] Putrescine dihydrochloride was purchased from GE Healthcare (Cat. No. CFA301), and the stock solution was diluted with distilled water to yield a radiological dose of 5 μCi/ml. Transglutaminase 2 was purchased from Sigma-Aldrich (Cat. No. T5398), and diluted with distilled water to yield a final concentration of 1 unit/ml. EGCG (Sigma-Aldrich, Cat. No. E4143) was dissolved in DMSO at a concentration of 10 mM to prepare its stock solution, and the stock solution was diluted with DMSO to prepare solutions having various concentrations.

450 μl of succinylated casein solution and 50 μl of [1,4-¹⁴C] putrescine dihydrochloride solution were mixed together to prepare a substrate solution. 96 μl of the reaction buffer, 3 μl of the stock solution of EGCG, and 1 μl of the stock solution of transglutaminase were mixed together to prepare each sample, followed by incubation at 37° C. for 10 min. 500 μl of the substrate solution and 100 μl of sample solution were mixed well, and the mixture was incubated at 37° C. for 2 hrs, before termination with 4.5 ml of cold (4° C.) 7.5% TCA. The final solution was stored at 4° C. for 1 hr. The TCA-precipitates were filtered through a GF/glass fiber filter, washed with 25 ml of cold 5% TCA, and dried. Radioactivity of crosslinked protein was measured using a (β-counter (Beckman Coulter), and compensated by the activity of DMSO-control group as a standard. The activity of transglutaminase was represented by the measured values. The assay was repeated three times under the same conditions, and the results are shown in the following Table 1.

TABLE 1

| | | | | | | SD (Standard |
|---|---|---|---|---|---|---|
| Concentration | | Assay 1 | Assay 2 | Assay 3 | Mean | deviation) |
| 0.0 | μM | 1.0000 | 1.0000 | 1.0000 | 1.0000 | 0.0000 |
| 0.5 | μM | 0.5166 | 0.5690 | 0.5295 | 0.5383 | 0.0273 |
| 1.0 | μM | 0.3950 | 0.3873 | 0.3207 | 0.3677 | 0.0408 |
| 2.0 | μM | 0.2009 | 0.2288 | 0.1707 | 0.2001 | 0.0291 |
| 3.0 | μM | 0.1911 | 0.1902 | 0.1482 | 0.1765 | 0.0245 |
| 5.0 | μM | 0.1246 | 0.0947 | 0.1000 | 0.1064 | 0.0159 |
| 10.0 | μM | 0.1029 | 0.0726 | 0.0881 | 0.0879 | 0.0152 |
| 50.0 | μM | 0.0861 | 0.0903 | 0.0849 | 0.0871 | 0.0028 |

In addition, the mean value was depicted in terms of concentration of EGCG. IC₅₀ values were calculated by a general nonlinear regression method, determined as 0.5169 (±0.0298) μM.

The relative inhibition activities of EGCG against transglutaminase are depicted in FIG. 1. As shown in FIG. 1, the activity of transglutaminase was inhibited in an EGCG concentration-dependent manner.

### INDUSTRIAL APPLICABILITY

The transglutaminase inhibitor which comprises EGCG as an active ingredient according to the present invention is safely applied to patients who suffer from the diseases caused by the overexpression of transglutaminase without causing side-effects, thereby being useful for the development of transglutaminase inhibitor.

1. A transglutaminase inhibitor, comprising epigallocatechin gallate (EGCG) or a derivative thereof.

2. A method of inhibiting an activity of transglutaminase using EGCG or a derivative thereof.

3. A pharmaceutical composition for the treatment or prevention of a disease caused by the activation of transglutaminase, comprising EGCG or a derivative thereof.

4. The pharmaceutical composition according to claim 3, wherein the disease is a neurological disease or cancer.

5. The pharmaceutical composition according to claim 4, wherein the neurological disease is selected from the group consisting of Alzheimer's disease, Huntington's disease, and Parkinson's disease.

6. A method for the treatment or prevention of a disease caused by the activation of transglutaminase, comprising the step of administering a substance selected from the group consisting of EGCG, a derivative thereof, and the pharmaceutical composition of any one of claims 3 to 5.

7. The method according to claim 6, wherein the disease is a neurological disease or cancer.

8. The method according to claim 7, wherein the neurological disease is selected from the group consisting of Alzheimer's disease, Huntington's disease, and Parkinson's disease.
